(19) United States
(12) Patent Application Publication
(10) Pub. No.: US 2026/0115443 A1
(43) Pub. Date: Apr. 30, 2026

(54) BONDED VOID VALVES, DEVICES INCLUDING SUCH VALVES, AND METHODS FOR MAKING SUCH VALVES

(71) Applicants: Stephen Arie Leeflang, Sandy, UT (US); Christian Scott Eversull, Palo Alto, CA (US)

(72) Inventors: Stephen Arie Leeflang, Sandy, UT (US); Christian Scott Eversull, Palo Alto, CA (US)

(21) Appl. No.: 19/376,879

(22) Filed: Oct. 31, 2025

Related U.S. Application Data
(60) Provisional application No. 63/714,807, filed on Oct. 31, 2024.

(51) Int. Cl. A61M 39/06; A61M 25/00; A61M 25/06; A61M 39/00

(52) U.S. Cl. CPC A61M 39/06; A61M 25/0097; A61M 25/0662; A61M 2039/0009; A61M 2039/062; A61M 2039/064; A61M 2039/0653; A61M 2205/0222; A61M 2205/58; A61M 2207/00

(57) ABSTRACT

Bonded void valves are provided for a sheath, catheter, or other tubular device that include a central valve portion comprising central mating surfaces opposite one another and a passage extending between the central mating surfaces along a valve axis; and first and second outer valve portions, each outer valve portion comprising an outer valve portion mating surface, an outer face opposite the outer valve portion mating surface, and a slit extending substantially parallel to the valve axis between the outer valve portion mating surface and the outer face, the outer valve portion mating surface of the first and second outer valve portions attached to respective central mating surfaces of the central valve portion such that the slits are aligned with the passage on opposite sides of the central valve portion.

This application claims benefit of co-pending U.S. provisional application Ser. No. 63/714,807, filed Oct. 31, 2024, the entire disclosure of which is expressly incorporated by reference herein.

### TECHNICAL FIELD

The application relates generally to apparatus and methods for delivering instruments and/or agents into a patient's body during a medical procedure, and, more particularly, to valves and hubs for guide sheaths, catheters, and other tubular devices for accessing body lumens and/or for delivering instruments into body lumens within a patient's body, and to methods for making and using them.

### BACKGROUND

There are many medical procedures where a guidewire, catheter, lead, and/or other medical device may be introduced into a patient's body cavity, recess, vessel, organ, and/or other body lumen. Such devices generally include one or more lumens/passages that extend from outside the patient's body into body passages or lumens, such as vessels (e.g., veins, arteries, lymph ducts, etc.) or chambers (e.g., heart, sinuses, etc.). Such devices almost always require one or more valves to prevent back bleeding and/or permit device introduction (e.g., guidewires, dilators, catheters, etc.) while preventing the loss of blood as well as ingress of air (or passage of other undesirable fluids or gases).

In many of these procedures, a delivery sheath, an access or guide catheter, or other tubular device including a valve on a proximal end thereof may be used to facilitate introducing medical devices into and through a lumen of the tubular device. It is generally desirable for the valve to provide a substantially fluid tight seal between the tubular device and other medical devices introduced therethrough, e.g., for the purpose of hemostasis, infusion of therapeutic or diagnostic agents, and the like.

With the advent of many large profile vascular access therapies (examples include percutaneous valve replacement systems, larger pulmonary vein isolation systems, and the like), the need for improved valve performance has been increasing. Current state-of-the-art valves generally perform well for smaller profile medical devices. For example, the demands on a valve in a smaller access device, e.g., to seal around medical devices having sizes up to 8.5 French (8.5 Fr; 2.8 mm), is much less as compared to an access device where the valve needs to seal over a range of devices up to fifteen French (15 Fr; 5.0 mm) or in some cases much higher. While the list of problems with state-of-the-art valves is long, the most significant problems are: 1) the inability to seal on a smaller device (such as a guidewire) after removal of a larger device (such as an ablation catheter), 2) the inability of the valve design to withstand prolonged use and wear (e.g., the valve may seal at the beginning of a procedure but not over time as multiple devices are removed and inserted), 3) high friction that is generally undesirable for the operator as well as contributing to valve wear and/or deterioration, and 4) complexity where multiple valve components are used serially and more complex operations are required.

In some cases, such as large bore access sheaths and deflectable sheaths, the performance can be extremely demanding including sealing at high pressures and low pressures over larger diameter devices and smaller diameter devices or when empty-all by a single valve used during the same procedure. It should also be noted that sealing under condition of vacuum is generally very important in order to prevent or reduce air ingress through the valve and potentially into the patient, e.g., into the cardiovascular system. As an example, a large bore deflectable sheath may be introduced over a dilator (having a diameter approximately equal to the sheath inner diameter) and a guidewire. The dilator may be removed leaving for a time only the guidewire through the valve. Subsequently, the sheath may be used to deliver an ablation catheter (of a size requiring the large bore deflectable sheath). The ablation catheter may include one or more baskets, spirals, splines, tines, etc. that must pass through the valve. After completing ablation, the catheter may be removed and much smaller mapping catheter(s) may be used to evaluate the ablation. Subsequently, the ablation catheter may be reinserted to touch up problem areas followed by additional mapping. An exchange over a guidewire may need to be performed and the valve must also seal on a small wire. Thus, in such a procedure, the valve must withstand repeated use, over a wide variety of device diameters and constructions, and must perform also in the empty condition-all without loss of seal.

Loss of seal in left sided cardiovascular procedures can result in myocardial infarction, stroke, or other complications from air ingress. The FDA Maude database continues to document a very large number of complaints on a wide variety of devices due to valve leakage—especially related to access sheaths and deflectable sheaths. State-of-the-art valves have not yet been able to meet these demanding requirements, as such a valve with improved sealing performance over large and small devices, at low and high pressures, as well as in the empty condition are of significant value.

By way of example, a cross-slit valve is a time-tested valve design used for many vascular access products that is simple and-in the case of smaller access devices effective. For such valves, a pair of slits may be provided that extend entirely through the valve from opposite sides, crossing through each other to create a point or line that acts a subsequent sealing surface for devices that are passed through the valve. In general, the cross-slit valve performs well in positive and negative pressure conditions with nothing passed through the valve (the faces of each slit seal on themselves under the deflection that comes during positive or negative pressure).

However, such valves usually fail when a large device is placed through that causes the sidewalls of the material of the passage between the two slits to stretch beyond their elastic limit. This may lead to tearing or plastic deformation of the valve material or both.

While the valve may continue to seal on the larger device (frequently not), it will not seal on smaller devices and, frequently, the tear prevents closure of the slits thus causing leaks in positive and negative pressure with no device positioned across the valve. Of very particular concern is air ingress, which may occur when a device is moved in or out or during aspiration from an access side port (a frequent occurrence for a host of reasons).

Thus, improved valves within a tubular access device, e.g., for sealing on small and large devices introduced through the valve and device, would be useful.

### SUMMARY

The present application is directed generally to apparatus and methods for delivering instruments and/or agents into a patient's body during a medical procedure. More particularly, the present application is related to valves and hubs for guide sheaths, catheters, and other tubular devices for accessing body lumens and/or for delivering instruments into body lumens of a patient's body, and to methods for making and using them.

Valve constructions have been previously disclosed related to “void valves,” such as those disclosed in U.S. Pat. No. 11,524,152, the entire disclosure of which is expressly incorporated by reference herein. The purpose of a void valve is to create a construction that separates the portion of the valve that seals over a device (the “void”) from the portion of the valve that seals when nothing is in it (slits), and where the void is, as the word void implies, in the middle of the valve. Such separation significantly reduces the propensity of tearing during use compared to state-of-the-art cross slit valves (the traditional state-of-the-art cross slit construction creates a cross point of effectively zero dimensional diameter by creating intersecting slits in a single valve body). As such, the passage of devices through such valves inevitably tears the valves as no valve material can have infinite stretch.

The creation of and the separation of a void within a valve may minimize or eliminate such risks of tearing and/or inadequate seal. Such constructions can be challenging to make and therefore valve constructions relying on this concept have been predominantly make in two parts, such as those disclosed in U.S. Pat. No. 11,524,152. Two-part construction has certain drawbacks, including the propensity for the two valve halves to bow and leave an open space between the valve halves, prop open the slits, etc.

In the present application, new valve constructions and methods for manufacturing such valves are provided. These take several forms, such as three-part bonded valves and two-part bonded valves, optionally with swelling lubricant.

For example, bonded two-part valves may be provided, i.e., with two valve portions bonded together to create a unitary valve construction. In one example, a two-part void valve may be provided in which both valve portions have substantially similar construction, e.g., including a portion of the void. One potential benefit of a two-part bonded construction is increasing the integrity of the void and preventing its (or the slits') opening, stretching, and shortening as may happen in non-bonded split valves during use. In another example, a single void valve may be provided, e.g., with one portion including a classic cross-slit therethrough bonded to a second portion including a full void or open passage therethrough. The valve portions (also referred to herein as “halves” even though they may not be identical or define true halves of the final valve) may be made of a range of materials, of most value are high consistency elastomer silicones that are compression molded, e.g., in 30 A, 40 A, and 50 A Durometers. Bonded constructions may include a “half” valve of one material and a “half” valve of another material.

Typically, the firmer (higher Durometer) material may be provided on the distal side of the valves, i.e., the valve portion on the inside of the valve relative to the patient (“inside” meaning inside the valve housing towards the inside of the patient, i.e., distal to the “outside”). The inside slit(s) on a valve generally seals against positive pressures, which may exceed thirty pounds per square inch (30 psi or 207 kPA), e.g. with infusion, while the outside slit(s) generally seals under negative pressure, which, absent use in a hyperbaric chamber, is limited to atmospheric pressure or approximately 14.7 psi (or 101 kPA). Alternatively, if the valve, and device including the valve, are expected to see relatively little internal pressure (such as body pressure only), the outside valve portion may be formed from firmer material than the inside valve portion. Other considerations including starting access through the valve may shift the balance to the outer valve portion being more towards the firmer material.

In one example, the valve halves may be physically identical (true “halves” having the same valve geometry bonded together on their void mating sides). In this example, both portions may include alignment features providing the ability to index the valve portions to a specific angular offset orientation, e.g., offset ninety degrees or other angles relative to each other. Alternatively, the valve halves may be asymmetrical, i.e., the valve halves may have different geometries and/or may be formed from different materials. Optionally, in either example, the geometries may include built in tapers and/or funnels on the non-mating face surfaces, e.g., for ease of introducing devices through the valve. Optionally, the non-mating sides (opposite the void in each valve half) may include one or more gussets or other features to further reinforce the closing property/ability of the slits, e.g., to enhance sealing the slits when no device is positioned through the valve.

In another example, bonded three-part valves may be provided that include proximal and distal portions, each including a slit, and a central portion including a full void or open passage therethrough, with the portions bonded together to create a unitary valve construction. Optionally, the non-mating sides (opposite the central portion) of the proximal and/or distal portions may include one or more gussets or other features to further reinforce the closing property/ability of the slits.

Any of the valve constructions herein may include swelling lubricant, i.e. lubricant that absorbs into the valve material, which may provide substantially continuous self-lubrication as well as swelling the sealing surfaces to further reinforce mating surfaces of other devices introduced through the valves. In one example, non-fluorinated silicone oil may be added to the valve, which may enhance lubrication of the valve as the oil may infiltrate molecular gaps in the material of the valve portions, e.g., silicone, thereby causing the valve material to swell. In addition or alternatively, topical lubricant, such as a fluorinated silicone oil, may be provided on surfaces of the valves that does not absorb into the valve material.

While the valve constructions provided herein are not limited to any particular materials (the valve constructions may enable improved performance even with lower performance materials), higher performing materials in valve construction are generally silicones. For example, the valves herein may be formed from silicone and, in one particular example, high consistency rubber (“HCR”) silicone materials, which may involve compression molding. HCR silicone materials may have the highest performance of all silicones in two key categories, i.e., elongation and tear resistance. Valve portions formed from such materials may be bonded together using silicone adhesives, such as RTV adhesive, UV activated films, and the like. Such adhesives may facilitate bonding to the silicone materials of the valve portions, and may enhance the ability of the adhesives to stretch with the valves.

In one exemplary method of bonding, a thin layer of adhesive may be dispersed or otherwise spread onto a transfer plate and the like, and the mating surface(s) of the valve portions (two-or three-part) may be touched or otherwise placed against the transfer plate, e.g., such that a thin but substantially uniform layer of adhesive is applied to the mating surface(s) without filling a void or other passages/recesses in the valve portions. This process may be comparable to an ink pad and stamp, i.e., where the ink is only substantially applied to the proud surfaces and not to the void. Such a method may ensure that the mating surfaces bond reliably but leave the void intact. Such transfer methods may also be highly controllable and scalable.

In accordance with another example, a valve housing including any of the valves herein may include a mechanism to snap and/or index a dilator or other device to the housing when the dilator fully inserted through the valve. Such mechanisms generally require an indexing feature larger than the dilator itself which means that the inlet of the housing allowing access to the valve must be significantly larger than the diameter of the dilator portion that is inserted through the valve. Given the relatively large inlet diameter, with conventional valve housings, it is possible for the user to attempt to introduce the dilator tip off the central axis of the valve thus potentially damaging the valve. Optionally, a valve housing may include a secondary element or structure, e.g., integrated into the valve housing or positioned within the valve housing adjacent the valve.

In one example, a valve companion funnel may be provided within a valve housing proximal to the valve, e.g., adjacent the inlet of the valve housing, that simply co-locates itself alongside the valve. The valve funnel or secondary element may include a recess offset in from the dilator hub indexing element just sufficiently that the indexing mechanism either just touches the valve companion funnel or is just proximally short. The valve companion funnel may include a passage having a diameter only nominally larger than the dilator diameter such that, upon introduction, the dilator tip is better centered to the central axis of the valve/device, reducing the likelihood of difficulty inserting or even damaging the valve. The valve companion funnel may be placed in or adjacent a valve hub bore and adjacent a valve, both of which may be secured by a cap.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1A is a cross-sectional side view of an example of a tubular device including a hub on its proximal end and a valve secured within the hub.

FIG. 1B is a cross-sectional side view of another example of a tubular device including a hub on its proximal end and a valve and secondary element or guide member secured within the hub.

FIG. 2A is an end view of an example of a bonded void valve that may be secured within the hub of FIG. 1 showing a pair of slits extending partially through the valve to a void within the valve.

FIG. 2B is a cross-sectional side view of the valve of FIG. 2A.

FIG. 2C is an exploded perspective view of the valve of FIGS. 2A and 2B, showing outer portions, each including a slit, that may be bonded on opposite sides of a central portion, including a void or passage therethrough.

FIGS. 3A and 3B are perspective and end views, respectively, of another example of a bonded void valve.

FIG. 3C is a partial cut-away view of the valve of FIGS. 3A and 3B showing the void within the valve.

FIG. 3D is an exploded perspective view of the valve of FIGS. 3A-3C, showing outer portions, each including a slit, that may be bonded on opposite sides of a central portion, including a void or passage therethrough.

FIG. 3E(1)-3E(3) are various views of an outer portion of the valve of FIGS. 3A-3C.

FIG. 3F(1)-3F(3) are various views of a central portion of the valve of FIGS. 3A-3C.

FIG. 4A is an exploded perspective view of another example of a valve, showing outer portions, each including a slit, that may be bonded on opposite sides of a central portion, including a void or passage therethrough.

FIG. 4B(1)-4B(3) are various views of an outer portion of the valve of FIG. 4A.

FIG. 4C(1)-4C(3) are various views of a central portion of the valve of FIG. 4A.

FIGS. 5A and 5B are cross-sectional and exploded cross-sectional views, respectively, of another example of a bonded void valve including two portions bonded together.

FIGS. 6A and 6B are exploded perspective and cross-sectional views, respectively, of an exemplary cap and valve funnel companion that may be included in a hub, such as the hub shown in FIG. 1B.

FIGS. 6C and 6D are end views of the cap of FIGS. 6A and 6B without and with the valve funnel companion received within the cap, respectively.

FIGS. 7A and 7B are perspective and end views of another example of a cap that may be provided on a hub, such as the hub shown in FIG. 1B.

FIG. 7C is a cross-section of the cap of FIGS. 7A and 7B.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

The term “substantially” is used throughout this document to indicate variations in the thus qualified terms. These variations are variations that do not materially affect the manner in which the invention works and can be due, for example, to uncertainty in manufacturing processes or to small deviations from a nominal value or ideal shape that do not cause significant changes to the invention. Also, the terminology “proximal” and “distal” refers to a position relative to an operator using the present disclosure on a patient. For example, distal elements are closer to an intervention site, e.g., within a patient's body, and proximal elements are closer to an operator, for example, a doctor or other operator using the devices herein.

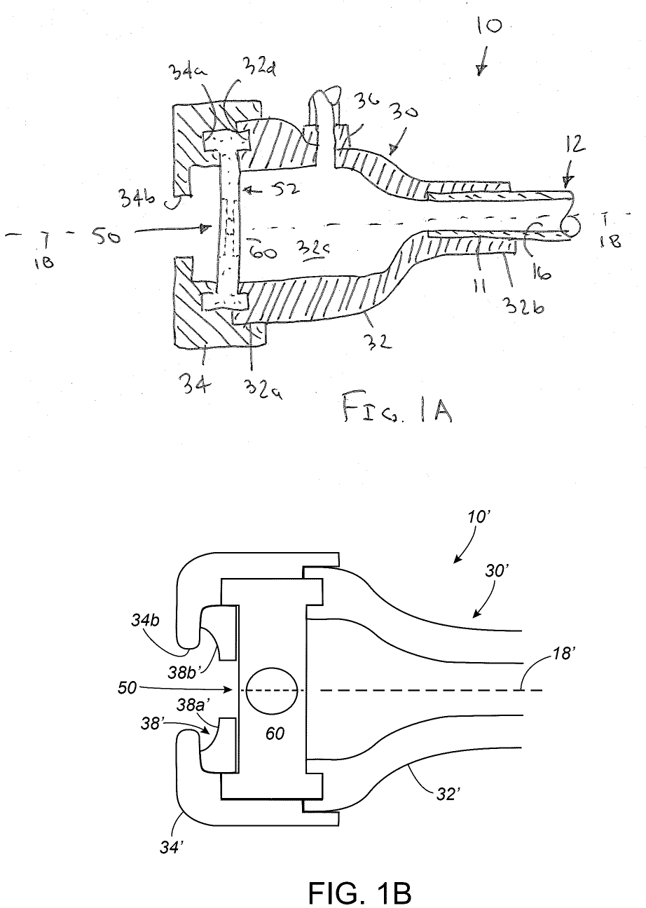

Turning to the drawings, FIG. 1A shows an example of an apparatus 10 for accessing a body lumen (not shown) and/or for delivering one or more fluids, agents, and/or instruments (also not shown) within a body lumen that may include a valve 50 according to any of the examples described herein. For example, the apparatus 10 may be a guide catheter, a procedure catheter, an access sheath, access port, or other tubular device sized for introduction into a body cavity, space, or lumen, such as a vessel within a patient's vasculature, a passage within a patient's gastrointestinal tract, urogenital tract, reproductive tract, respiratory tract, lymphatic system, a potential space, such as the pericardial space, and the like.

Generally, the apparatus 10 includes an elongate tubular body 11 including a proximal end 12, a distal end sized for introduction into a body lumen (not shown), a lumen 16 extending between the proximal end 12 and distal end along a central longitudinal axis 18, and a handle or hub 30 on the proximal end 12 including a valve 50 for allowing one or more devices to be introduced and/or fluids to be infused into the lumen 16. Optionally, the apparatus 10 may include one or more additional lumens (not shown), which may be disposed concentrically around, side-by-side with, or otherwise adjacent the lumen 16. The lumen 16 may be sized for receiving a guide wire, procedure catheter, cardiac lead, needle, or other instrument (not shown), and/or for delivering fluids or other flowable agents or materials therethrough, e.g., as described elsewhere herein.

The tubular body 11 may be constructed from one or more layers, e.g., an inner liner surrounding the lumen 16, a reinforcing layer surrounding the inner liner, and an outer layer (not shown). Optionally, one or more coatings (also not shown) may be applied to the inner surface of the inner liner. In one example, a hydrophilic coating, such as Polyvinylpyrrolidone, may be sprayed or otherwise applied onto the surface of the inner liner during fabrication to provide a lubricious inner surface for the lumen 16 of the tubular body 11. The layers of the tubular body 11 may be attached to one another, e.g., by one or more of laminating, adhering, adhesive bonding, ultrasonic welding, reflowing or other heating, and the like. Exemplary materials and methods for making the tubular body 11 are disclosed in U.S. Pat. Nos. 7,553,387, 7,550,053, 8,673,100, and 8,454,578, the entire disclosures of which are expressly incorporated by reference herein.

The construction of the tubular body 11 may be substantially uniform or may vary between the proximal and distal ends, e.g., by varying the construction of the inner liner, reinforcing layer, and/or outer layer along the length of the tubular body 11. For example, the outer layer at or adjacent the proximal end 12 may be substantially rigid or semi-rigid, e.g., providing sufficient column strength to facilitate the apparatus 10 being pushed and/or otherwise manipulated from the proximal end 12. In addition, the reinforcing layer or the material of the outer layer may allow the apparatus 10 to be twisted from the proximal end 12, e.g., to rotate the distal end within a patient's body. Thus, the distal end of the apparatus 10 may be manipulated within a patient's body from the proximal end 12 without substantial risk of buckling and/or kinking. Optionally, the outer layer at or adjacent the distal end may be substantially flexible or semi-rigid, e.g., to allow the distal end to bend easily or otherwise be advanced through tortuous anatomy and/or provide a substantially atraumatic distal tip. Exemplary outer layers and constructions that may be included in the apparatus 10 and methods for making them are disclosed in U.S. Pat. Nos. 4,478,898, 4,863,442, 5,217,440, 5,254,107, 5,676,659, 5,811,043, 5,836,926, 6,004,310, 6,669,886, 6,837,890, and 6,945,970, the entire disclosures of which are expressly incorporated by reference herein.

In various examples, the tubular body 11 may have an outer diameter between about half and twenty millimeters (0.5-20 mm) or between about one and five millimeters (1-5 mm), and a length between about five and one hundred fifty centimeters (5-150 cm). The inner liner may have a wall thickness between about 0.0001-0.01 inch (0.0025-0.25 mm) and the outer layer may have a wall thickness between about 0.0005-0.2 inch (0.0127-5.08 mm).

With continued reference to FIG. 1A, an example of a hub 30 is shown that includes a first hub portion, e.g., hub body 32, and a second hub portion, e.g., cap 34, which may be coupled together, e.g., to secure a valve 50 within or to the hub 30. As shown, the hub body 32 may be an elongate tubular body including a first end 32a, a second end 32b, and a hub lumen 32c extending therebetween that communicates with the lumen 16 of the tubular body 11. The cap 34 may be connected to the first end 32a, e.g., using one or more of cooperating connectors, bonding with adhesive, sonic welding, fusing, interference fit and the like, to permanently (or alternatively, removably) capture the valve 50 within the hub 30.

For example, as shown in FIG. 1A, the hub body 32 and cap 34 may include annular recesses 32d, 34a that receive a flange 70 on a perimeter of the valve 50, as described elsewhere herein. Alternatively, a valve may be provided without a flange and a perimeter of the valve may be captured by and/or otherwise secured between a hub body and cap of the hub (not shown). Additional configurations of hubs that may be used with the valves provided herein may be found in U.S. Pat. No. 11,524,152.

Returning to FIG. 1A, the second end 32b of the hub body 32 may include one or more connectors (not shown) for attaching the tubular body 11 to the hub 30. For example, the second end 32b may include one or more tabs, slots, threads, and the like (not shown) that may be engaged with complementary slots, tabs, threads, and the like (also not shown) on the proximal end 12 of the tubular body 11. In addition or alternatively, the second end 32b may be engaged with the proximal end 12 by one or more of an interference fit, attached by bonding with adhesive, fusing, sonic welding, heat bonding, reflowing, insert molding, and the like, as desired.

Optionally, the hub body 32 may also include one or more side ports, e.g., a first side port 36 communicating with the lumen 32c, e.g., to allow a source of fluid, e.g., a syringe, pump, air/vacuum line, and the like (not shown), to be coupled to the hub 30, e.g., to deliver fluid into the lumen 16 of the tubular body 11 and/or aspirate fluid from the lumen 16.

Optionally, the cap 34 may include a transition, guide, or other features (not shown) at the opening 34b to guide and/or otherwise facilitate directing devices into the hub 30 and lumen 16 of the tubular body 11, such as the guide member 38′shown in FIG. 1B and described further elsewhere herein.

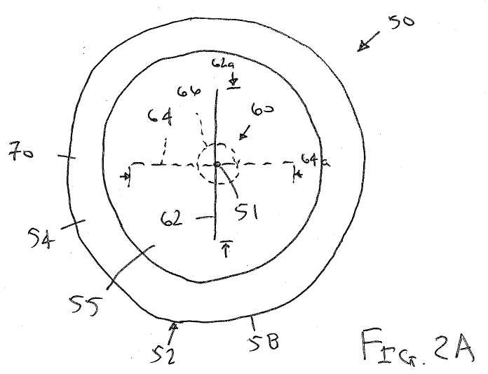

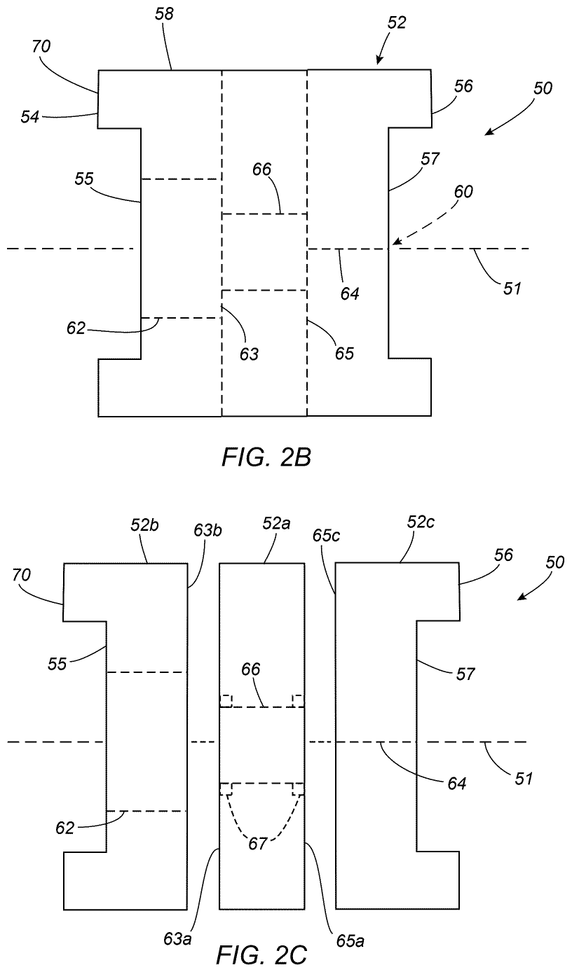

Turning to FIGS. 2A-2C, an example of a valve 50 is shown that may be received in the hub 30 shown in FIG. 1A. Generally, the valve 50 includes a valve body 52 including a proximal, inlet, or first end 54, a distal, outlet, or second end 56, an outer surface 58 extending between the first and second ends 54, 56, and a valve passage 60 extending between the first and second ends 54, 56, e.g., along a valve axis 51. As explained elsewhere herein, the passage 60 is configured to provide a substantially fluid-tight seal when a medical device (not shown) is inserted into the valve 50 while also providing a fluid-tight seal when the valve passage 60 is empty (i.e., without a medical device inserted into the valve passage 60).

As best seen in FIG. 2B, the valve body 52 includes a first face 55 adjacent the first end 54, and a second face 57 opposite the first face 55, i.e., adjacent the second end 56, with the passage 60 extending from the first face 55 to the second face 57. The faces 55, 57 may have a planar and/or substantially circular shape, e.g., extending across a cross-section of the valve body 52, e.g., within the perimeter of the outer surface 58, as shown in FIG. 2A, or may have another desired shape, e.g., and oblong shape, and the like (not shown).

Optionally, the valve 50 may include an annular flange 70 extending around the perimeter of the valve body 52, e.g., surrounding the faces 55, 57. The flange 70 may have a thickness (i.e., dimension parallel to the valve axis 51) that is greater than the distance between the faces 55, 57, as shown in FIGS. 1A and 2B. Alternatively, the flange 70 may be omitted and the valve body may have a uniform thickness within the outer surface 58, i.e., between the faces 55, 57 (not shown).

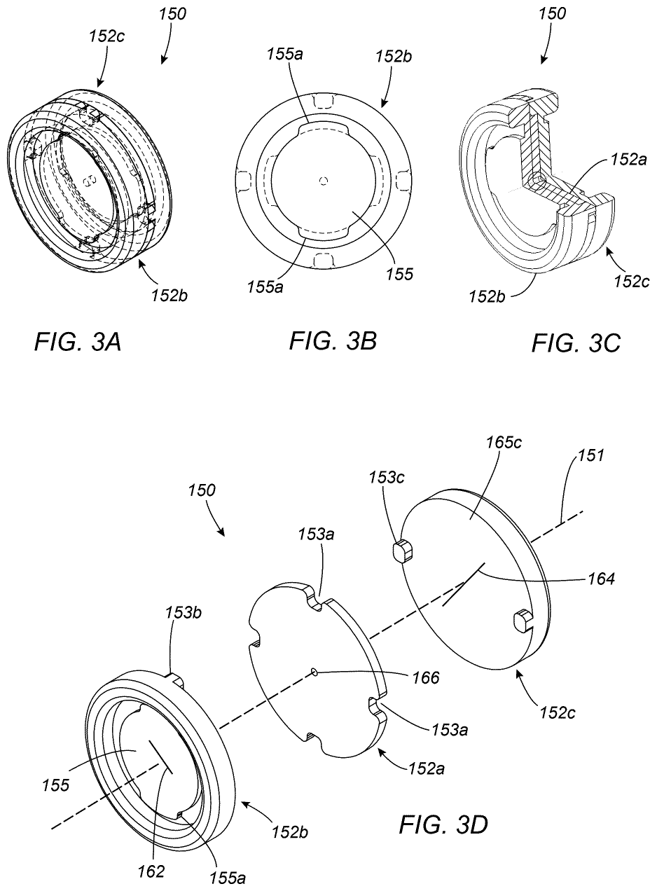

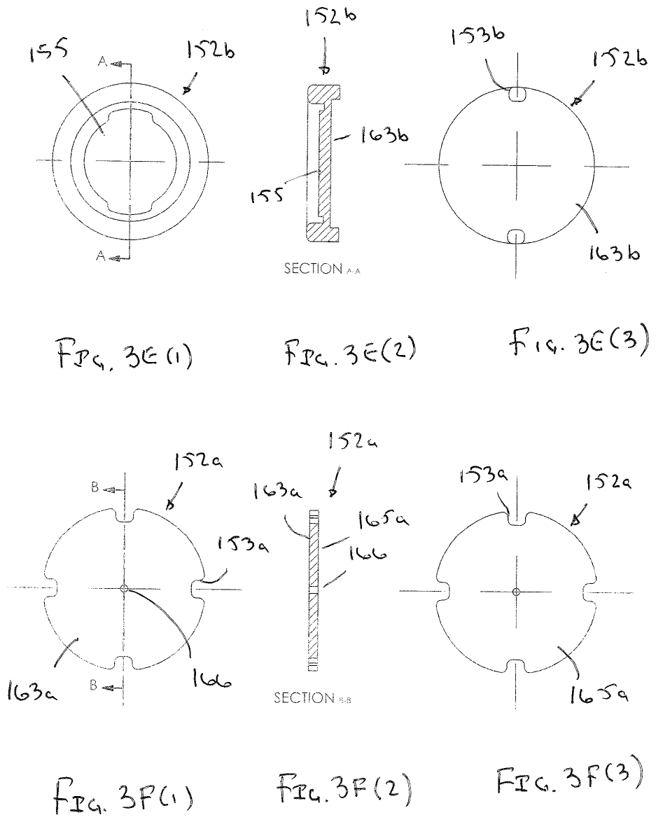

In a further alternative, a relatively thin intermediate annular region may be provided in the valve body (not shown), e.g., with thickness being aligned along the axis 51, such that a relatively thicker central region includes the passage 60 and is surrounded by the relatively thin intermediate region, and a relatively thicker outer perimeter is provided around the intermediate region, e.g., similar to the valve 150 shown in FIGS. 3A-3D, and described further elsewhere herein.

In the example shown in FIGS. 2A and 2B, the faces 55, 57 are substantially planar, e.g., extending substantially perpendicular to the axis 51 of the valve 50. Alternatively, the faces may have other non-planar shapes if desired (not shown), e.g., similar to the valves shown in U.S. Pat. No. 11,524,152.

Returning to FIGS. 2A and 2B, the passage 60 generally includes a first slit 62, a second slit 64, and an open central bore or passage 66. For example, the first slit 62 may extend substantially parallel to the valve axis 51 from the first face 55 towards the second face 57 to a first intermediate location 63 within the valve body 52. Similarly, the second slit 64 extends substantially parallel to the valve axis 51 from the second face 57 towards the first face 55 to a second intermediate location 65 within the valve body 52. The central bore 66 extends along the valve axis 51 between the first intermediate location 63 and the second intermediate location 65. Thus, the central bore 66 may provide an interior or void region that may accommodate medical devices introduced through the passage 60, e.g., expanding while providing a contact surface against the devices to maintain a fluid-tight seal, while the slits 62, 64 may enhance sealing the passage 60 when the devices are removed from the valve 50, e.g., resiliently closing such that opposite surfaces of the slits 62, 64 contact one another to seal the passage 60.

As shown in FIG. 2B, the central bore 66 may have a substantially uniform circular diameter extending between the intermediate locations 63, 65. For example, with the valve body 52 in a relaxed state or empty condition (e.g., without a medical device inserted into the bore 62), the central bore 66 may have a diameter or other maximum cross-section between about 0.25 and eight millimeters (0.25-8 mm), e.g., not more than about 0.030 inch (0.75 mm), but may be expandable to larger diameters, e.g., between about 0.35 and ten millimeters (0.35-10 mm). In one particular example, the central bore 66 may have a substantially uniform circular cross-section, e.g., having a substantially uniform diameter between about 0.020-0.030 inch (0.5-0.75 mm), along its axial length. Alternatively, the bore may have other shapes, e.g., a uniform non-circular cross-section extending between the intermediate locations (not shown).

As shown in FIG. 1A, the valve axis 51 of the valve 50 may be aligned with the longitudinal axis 18 of the hub 30 and tubular body 11 when the valve 50 is positioned within the hub 30, e.g., such that the passage 60 is centered within the hub 30. Alternatively, the axis of the passage 60 within the valve 50 may be offset from the longitudinal axis 18, e.g., such that the slits and bore are located closer to the outer surface of the valve body on one side than the opposite side (not shown). Consequently, in this alternative, a side wall region of the valve body may be thinner on one side of the valve than other side wall regions, which may facilitate slitting the valve during use along the thinner region.

Returning to FIGS. 2A and 2B, the valve body 52 may be formed from an elastomeric material, such as silicone (e.g., having a durometer between about 30 A-50 A), chronoprene, isoprene, santoprene, polyurethane, other elastomeric materials, and the like. Alternatively, other flexible synthetic or natural materials may be used for the valve body 52, e.g., thermoplastics, thermoset plastics, natural and synthetic rubbers, and the like. Within silicones, there are a range of LSR (liquid silicone rubber) material as well as HCR silicones (high consistency rubber) that may be used. The valve body 52 may be formed from material having substantially uniform construction and/or mechanical properties. Alternatively, the valve body 52 may be formed from multiple materials, e.g., having different mechanical properties, e.g., Durometer, at different locations along the valve axis 51.

With additional reference to FIG. 2C, the valve 50 may be constructed from multiple portions, e.g., a central portion 52a and first and second outer portions 52b, 52c, which may be formed separately and then bonded or otherwise attached together to provide a unitary construction. For example, as shown, the central portion 52a includes mating surfaces 63a, 65a opposite one another and the central bore 66 extends entirely through the central portion 52a along the valve axis 51 between the mating surfaces 63a, 65a. The first and second outer portions 52b, 52c may be substantially identical to one another, e.g., as shown in FIG. 2C, or may have different geometries and/or material constructions. As shown, each outer portion 52b, 52c includes an inner mating surface 63b, 65c, an outer face 55, 57 opposite the inner mating surface 63b, 65c, and a slit 62, 64 extending substantially parallel to the valve axis 51 between the mating surfaces 63b, 65c and the outer faces 55, 57.

The inner mating surfaces 63b, 65c of the outer portions 52b, 52c are bonded or otherwise attached to respective mating surfaces 63a, 65a of the central portion 52a, e.g., such that the slits 62, 64 are aligned with the central bore 66 on opposite sides of the central portion 52a. As shown, the outer portions 52b, 52c may be rotated around the valve axis 51 relative to one another before being bonded to the central portion 52a, e.g., such that the slits 62, 64 are rotationally offset from one another around the valve axis 51. In the particular example shown, the slits 62, 64 may be orthogonal to one another, i.e., offset about ninety degrees (90°) around the valve axis 51. For example, the first slit 62 may have a first width extending across a portion of the first face 55 and the second slit 64 may have a second width across a portion of the second face 57 such that the second width is offset around the valve axis from the first width and the two widths intersect at the valve axis 51.

In an exemplary method, adhesive may be applied to one or both of the mating surfaces 63a-63b and 65a-65c to bond the portions 52-52c together. For example, adhesive may be dispersed or otherwise spread on a surface of a transfer plate or other device (not shown), and then the desired mating surfaces may be touched against the transfer plate to apply a thin, e.g., substantially uniform, layer of adhesive to the mating surface(s). For example, this method may be used to apply a layer of adhesive to the central portion mating surfaces 63a, 65a without applying adhesive that may partially or entirely obstruct the central bore 66. The adhesive may be applied to both mating surfaces that are to be bonded together or to only one of the mating surfaces, as desired.

Optionally, as shown in FIG. 2C, an annular recess 67 may be provided around the central bore 66 that extends axially from one or both mating surfaces 63a, 65a into the central portion 52a. Such recess(es) 67 may provide a region of the central portion 52 surrounding the central bore 66 that is not bonded to the axially adjacent material of the outer portions 52b, 52c, which may facilitate initial expansion of the central bore 66 as a device is introduced through the valve 50 that has a diameter or other cross-section larger than the central bore 66 in its relaxed state.

The portions 52a-52c may be formed from the same or different materials, generally elastomeric materials such as silicone. In one particular example, the portions 52a-52c may be formed from high consistency rubber (“HCR” silicone), which may enhance elongation and/or tear resistance, e.g., when relatively larger devices are introduced through the central bore 66 and cause the material to dilate outwardly. For such silicone materials, it may be desirable to use silicone adhesives, such as RTV adhesive, UV activated films, and the like, to bond the portions together.

Each of the portions 52a-52c may be formed using conventional methods, e.g., by compression molding, casting, machining, laser cutting, and the like. The central bore 66 may be cut or otherwise created through the central portion 52 after molding or, alternatively, the central bore 66 may be created when the central portion is formed. For the outer portions 52b, 52c, the slit 62, 64 may be cut or otherwise created through the outer portions 52b, 52c after the outer portions 52b, 52c are molded or otherwise formed. The slits 62, 64 have corresponding widths across the first and second faces 55, 57, respectively, which may be substantially the same or, alternatively, one of the widths may be longer than the other. Generally, the widths of the slits 62, 64 may be sufficient to allow opposing regions of the valve body 52 on either side of the slit 64 to be directed away from one another to accommodate receiving a medical device through the passage 60 without substantial risk of the valve body 52 tearing uncontrollably.

Returning to FIGS. 2A and 2B, the resulting valve body 52 after bonding the portions 52a-52 together may provide a central bore 66, which may be dilated or otherwise expanded when a medical device is inserted into the bore 66 and may resiliently return to its original size when the medical device is removed. Thus, the bore 66 may expand to accommodate a medical device having a larger cross-section than the bore 66 with the valve body 52 in the relaxed state.

Similarly, the flexibility of the material may allow the sides of the slits 62, 64 to separate to accommodate introducing a device through the passage 60 and then the sides may resiliently close once the device is removed. Thus, the valve body 52 may be resiliently flexible such that the opposing slit regions may expand when a medical device is inserted through the slits 62, 64, yet resiliently close when the medical device is removed to maintain a substantially fluid tight seal through the valve passage 60.

Optionally, e.g., in the case of silicone valves, the valve may be lubricated with silicone or other lubricant that may absorb into the valve providing some self-lubricating properties, e.g., non-fluorinated silicone. Additionally, or alternatively, for example, a lubricant that will not absorb into the base material may be used, such as a fluorinated silicone lubricant. In the case where both lubricants are used, the lubricant available on the surface may be useful for priming a dilator initially introduced through the valve. Such dilators are often the largest and “stickiest” device to go through a valve capable of applying the greatest stress to the valve. Thereafter, absorbed lubricant may continue to lubricate other devices as the surface lubricant is used up.

The valve 50 may be mounted within a hub 30 of a tubular device 10, e.g., secured between a hub body 32 and cap 34, as shown in FIG. 1A. As shown, the cap 34 may include a lip defining an inlet or opening 34b into which one or more devices (not shown) may be inserted to introduce the devices through the valve 50, i.e., through the slits 62, 64 and central bore 66, and into the lumen 16 of the tubular device 10. In one example, the lip may be sized to capture an indexing feature, locking feature, and the like on a secondary device inserted through the inlet 34b, e.g., to prevent subsequent axial movement of the secondary device. For example, a dilator may include an indexing feature, e.g., an annular ridge (not shown), on its proximal end that is larger than a distal portion of the dilator that is introduced through the valve 50 and lumen 16, which may be captured distally behind the lip, e.g., to allow the device 10 and dilator to be introduced together into a patient's body.

Optionally, as shown in FIG. 1B, a hub 30′ may be provided that includes a valve companion funnel or other secondary element 38′ within the hub 30′ proximal to the valve 50, e.g., adjacent the inlet 34b′ of the cap 34′, that co-locates itself alongside the valve 50. The secondary element 38′ may include a recess offset in from the dilator indexing element sufficiently that the indexing mechanism either just touches the valve companion funnel 38′ or is just proximally short. As shown, the valve companion funnel 38′ may include a passage 38a′ having a diameter only nominally larger than the dilator diameter such that, upon introduction, the dilator tip is better centered to the longitudinal axis 18′, reducing the likelihood of difficulty inserting or even damaging the valve 50. In addition, the valve companion funnel 38′ may include a tapered or ramped proximal surface 38b′, which may guide the dilator tip and/or otherwise facilitate aligning the dilator tip with the passage 60 through the valve 50. Similarly, the valve companion funnel 38′may be sized or formed to guide other devices into and through the valve 50, for example basket or other complex shaped catheters which must compress or deform to pass the valve 50. The valve companion funnel 38′may be formed from a variety of rigid or semi-rigid material, e.g., by one or more of molding, casting, machining, laser cutting, printing, and the like. During manufacturing and/or assembly of the hub 30,′ the valve companion funnel 38′ may be placed in the hub body 32′ adjacent the valve 50, and both may be secured within the hub body 32′ by the cap 34.′

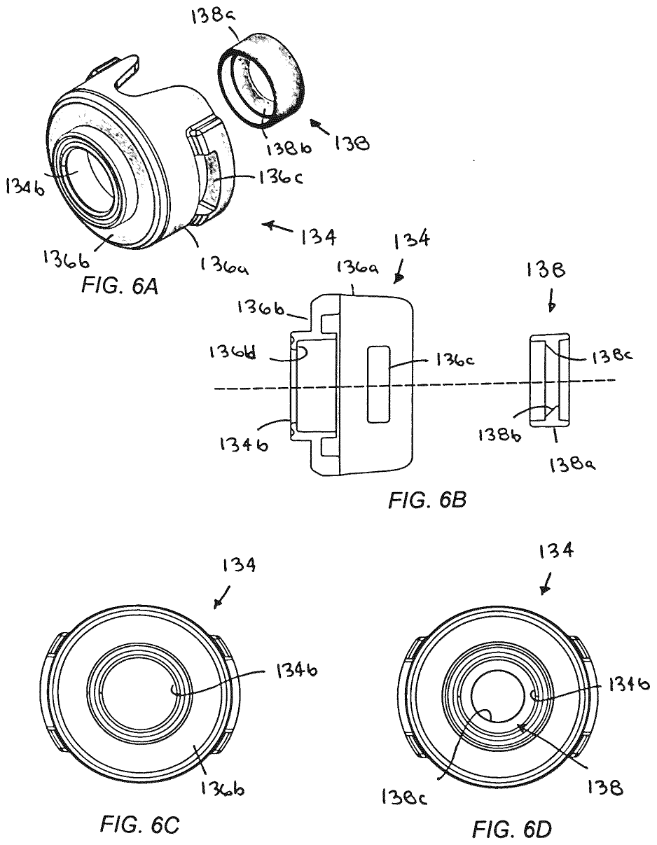

Turning to FIGS. 6A-6D, another example of a cap 134 and guide member 138 are shown, which may be coupled to a hub (not shown), similar to the hub 30.′ The cap 134 may include a sidewall 136a, which may be received over or otherwise connected to a proximal end of a hub body (not shown) and an end wall 136b that includes opening 134b. The sidewall 136a may include one or more connectors, e.g., a pair of slots and/or detents 136c that may engage with corresponding connectors on the hub body (not shown) to secure the cap 134 after inserting a valve (also not shown) and the guide member 138 within the hub body. The end wall 136b may include a lip or flange 136b surrounding the opening 134b, thereby providing a pocket 136d distal to the opening 134b within which an indexing or locking element of a dilator or other medical device (not shown) may be received when the medical device is introduced through the guide member 138 and valve.

As shown in FIGS. 6A and 6B, the guide member 138 includes a ring or other annular body 138a surrounding a passage therethrough that includes a ramped or tapered surface 138b surrounding an outlet 138c having a smaller diameter than the opening 134b in the cap 134. Consequently, when a distal tip of a dilator or other medical device is introduced into the hub, the distal tip may be guided by the surface 138b through the outlet 138c, thereby centering the distal tip as it then enters the valve.

During assembly, the guide member 138 may be inserted into the hub body after the valve, and then the cap 134 may be attached to the proximal end of the hub body to secure the guide member 138 and valve within the hub, similar to other devices herein.

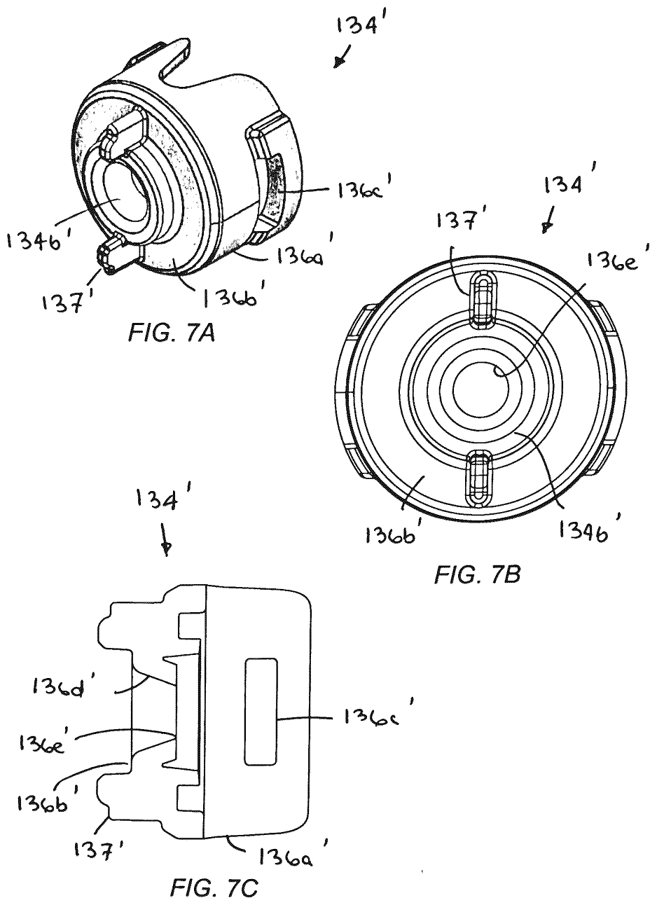

Alternatively, turning to FIGS. 7A-7C, a cap 134′ may be provided that includes an integral cap ring on the end wall 136b′ that includes an inlet 134b', which may facilitate guiding a secondary device through the cap 134′ into a valve (not shown) without a separate guide member. As shown, the cap ring includes a ramped or tapered surface 136d′ tapering inwardly from the inlet 134b′ to an outlet 136e′ that may be aligned along the valve axis of the valve (not shown) to guide a distal tip of a medical device into the valve. Optionally, the cap 134′ may include one or more locking features, e.g., a snap feature, to engage corresponding features on the secondary device. For example, as shown, the cap 134′ may include a plurality of tabs 137′ extending proximally from the end wall 136b', e.g., a pair of tabs 137′ on opposite sides of the inlet 134b'. A snap lock cap (not shown) may be attached to the hub that includes slots (not shown) that receive the tabs 137′ such that the cap lock may move radially outwardly away from the inlet 134b′ to accommodate an indexing or locking element on a medical device passing distally through the snap lock into the cap 134′. The cap lock may resiliently return radially inwardly to capture the indexing or locking element to secure the medical device relative to the hub.

Turning to FIGS. 3A-3D, another exemplary valve 150 is shown that includes a central valve portion 152a and first and second outer valve portions 152b, 152c, which may be formed separately, e.g., by one or more of molding, casting, machining, laser cutting, and the like, whereupon the mating surfaces of the portions 152a-153c may be positioned against and/or attached to one another to provide a passage 160 extending between opposite faces 155 of the final valve 150, generally similar to valve 50.

With additional reference to FIG. 3F(1)-3F(3), the central portion 152a includes mating surfaces 163a, 165a opposite one another and a central bore or passage 166 that extends entirely through the central portion 152a along valve axis 151 between the mating surfaces 163a, 165a. With additional reference to FIG. 3E(1)-3E(3), each of the outer portions 152b, 152c includes an outer face 155 and a mating surface 163b, 165c opposite one another, and a slit 162, 164 extending substantially parallel to the valve axis 151 from the outer face 155 to the mating surface 163b, 165c.

Optionally, one or both faces 55, 57 may include a cross-section that may engage with a cap and/or hub body when the valve 50 is loaded into a hub, e.g., to enhance sealing and/or support the valve 50 when a medical device (not shown) is introduced through the passage 60. For example, an outer face 155 of one or both outer portions 152b and/or 152c may include a feature(s), that may engage with a cap or hub body, e.g., during assembly of a tubular device including the valve 150. For example, a cap or hub body may have a non-circular shape or feature extending radially inward (not shown) such that, when the valve is axially compressed within the cap or hub body during assembly, the shape, feature, and/or valve material may be radially compressed inwardly, which may support the material adjacent the slit(s) 162, 164 and/or direct the slit(s) 162, 164 to a closed condition.

With particular reference to FIG. 3D, unlike the valve 50, the valve portions 152a-152c include one or more alignment or interlocking elements for aligning the portions 152a-15c together in a predetermined orientation. For example, as shown in FIGS. 3D and 3E(3), each outer portion 152b, 152c includes a pair of tabs 153b, 153c extending from the mating surfaces 163b, 165c, e.g., axially towards the central portion 152a, and the central portion 152a includes corresponding recesses 153a sized to receive respective tabs 153b, 153c. In the example shown, the pair of tabs 153b, 153c are located adjacent the outer perimeter of the outer portions 152b, 152c, e.g., positioned across from one another on opposite sides of the slits 162, 164 and axis 151 on the mating surface 163b. The central portion 152a include two pairs of recesses 153a that are similarly positioned adjacent the outer perimeter of the central portion 152a. The pairs of recesses are offset from one another around the outer perimeter such that the outer portions 152b, 152c need to be rotationally offset relative to one another to allow the respective pairs of tabs 153b, 153c to be received in the corresponding recesses 153a.

Thus, during assembly, the outer portions 152b, 152c are rotationally oriented to insert the tabs 153b, 153c into the respective recesses 152a, thereby rotationally orienting the slits 162, 164 relative to one another. Optionally, as shown in FIG. 3C, at least one of the outer portions 152c may include an annular ridge or sized to be positioned over the outer perimeter of the central portion 152a when the valve 150 is assembled. As with the valve 50, the portions 152a-152c may be bonded or otherwise attached together to provide the final valve 150.

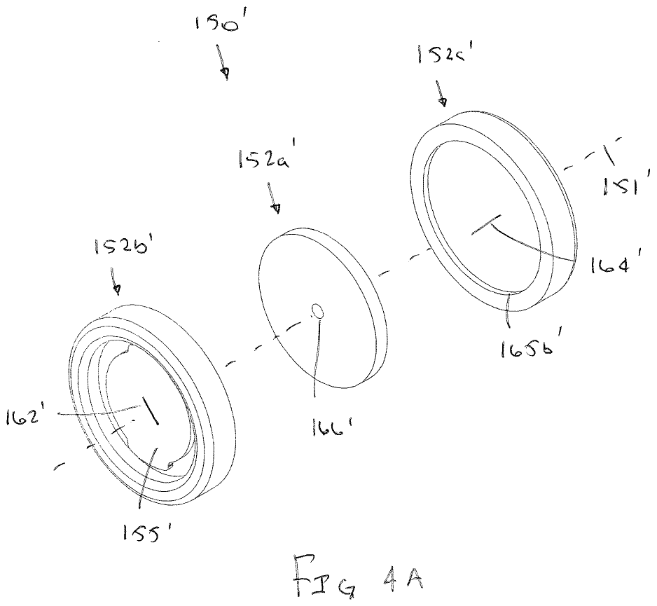

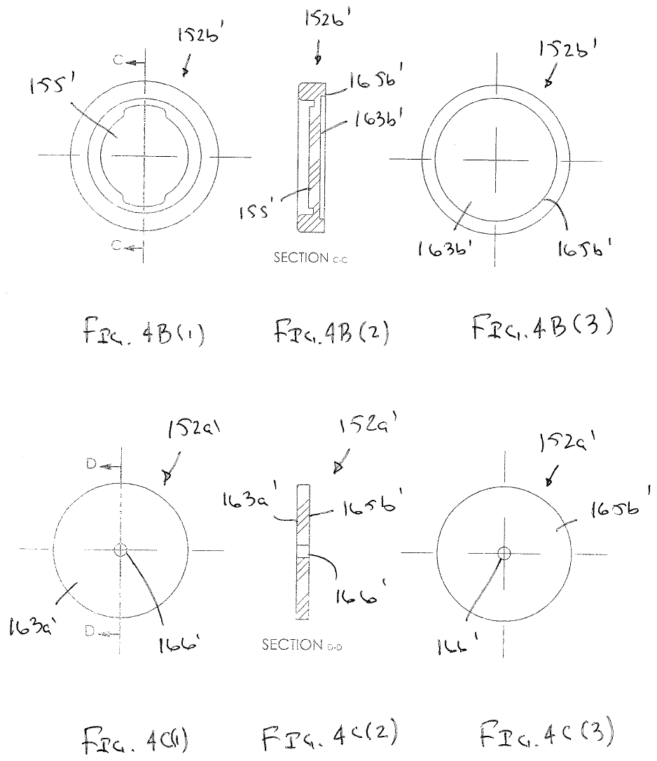

Alternatively, as shown in FIGS. 4A-4C, a valve 150′may be provided that does not include alignment elements. For example, as shown in FIG. 4C(1)-4C(3), a central portion 152a′ may simply be a circular disc including opposite mating surfaces 163a', 165a′ and a central bore 166′ therethrough. At least one of the outer portions 152b′ includes an annular ridge 165b′ surrounding the mating surface 163b,′ which may be sized to surround the outer perimeter of the central portion 152a.′ Thus, the central portion 152a′ may simply be sandwiched between and bonded to the outer portions 152b,′ 152′without requiring a particular rotational orientation of the portions 152a'-152c.′ The annular ridges at the outer perimeters of the outer valve portions 152b', 152c′ may contact one another adjacent the outer perimeter of the central portion 152a′ (and optionally may be bonded to one another) or, alternatively, the annular ridges may be spaced apart from one another, thereby providing a gap between the outer portions 152b', 152c′ around the central portion 152′ captured between the outer portions 152b', 152c'.

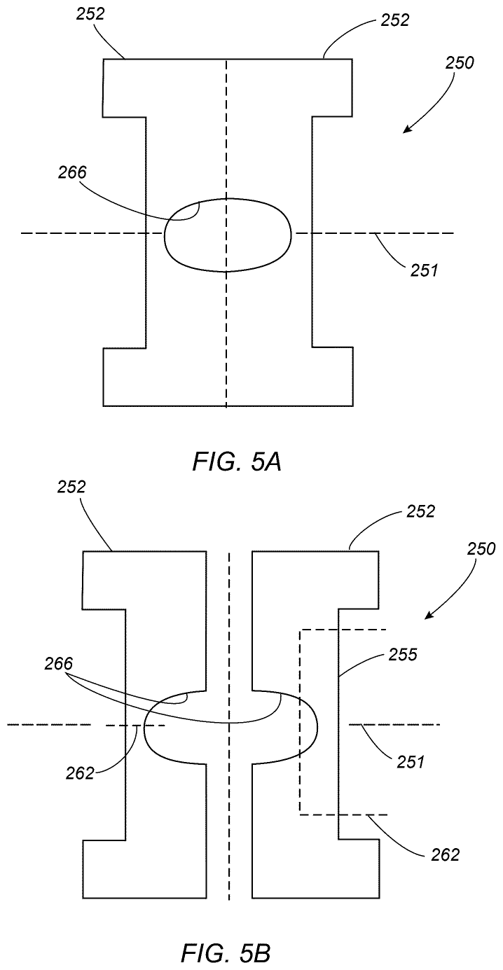

Turning to FIGS. 5A and 5B, another example of a bonded void valve 250 is shown that includes two valve portions 252 that each includes a slit 262 extending from an outer face 255 partially towards an inner mating surface 263. Each valve portion 252 also includes a void or central bore 266 extending from the mating surface 263 partially towards the outer face 255. The slit 262 and bore 266 are axially aligned with one another to provide a passage extending through the portions 252. Similar to other valves herein, the valve portions 252 may be rotationally oriented relative to one another before bonding to offset the slits 262 around a valve axis 251 of the valve 250.

The foregoing disclosure of examples has been presented for purposes of illustration and description. It is not intended to be exhaustive or to limit the invention to the precise forms disclosed. Many variations and modifications of the examples described herein will be apparent to one of ordinary skill in the art in light of the above disclosure.

Further, in describing representative examples, the specification may have presented the method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.
This listing of claims replaces all prior versions and listings of claims in the application.

1. A valve for a hub of a sheath, catheter, or other tubular device, comprising:
a central valve portion comprising central portion mating surfaces opposite one another and a passage extending between the central portion mating surfaces along a valve axis; and
first and second outer valve portions, each outer valve portion comprising an outer valve portion mating surface, an outer face opposite the outer valve portion mating surface, and a slit extending substantially parallel to the valve axis between the outer valve portion mating surface and the outer face;
the outer valve portion mating surface of the first and second outer valve portions attached to respective central portion mating surfaces of the central valve portion such that the slits are aligned with the passage on opposite sides of the central valve portion.

2. The valve of claim 1, wherein the first and second outer valve portions and the central valve portion comprise one or more alignment elements for rotationally aligning the first and second outer valve portion mating surfaces to the central valve portion in a predetermined orientation.

3. The valve of claim 2, wherein the first slit has a first width across a portion of the first face and the second slit has a second width across a portion of the second face, and wherein the alignment elements orient the first and second slits such that the second width is offset around the valve axis from the first width.

4. The valve of claim 3, wherein the first and second widths are orthogonal to one another around the valve axis.

5. The valve of claim 2, wherein the one or more alignment features comprise a tab extending from the outer valve portion mating surface of each of the first and second outer valve portions and an opening in the central valve portion that receives each tab.

6. The valve of claim 5, wherein the one or more alignment features comprise a pair of tabs extending from the outer valve portion mating surface of each of the first and second outer valve portions on opposite sides of the valve axis and openings in the central valve portion that receive respective tabs of the pair of tabs to orient the outer valve portions relative to the central portion.

7. The valve of claim 1, wherein the first and second outer valve portion mating surfaces are permanently attached to the respective mating surfaces of the central portion.

8. The valve of claim 7, wherein the first and second outer valve portion mating surfaces are bonded to the respective mating surfaces of the central portion.

9. The valve of claim 8, wherein the first and second outer valve portion mating surfaces are bonded to the respective mating surfaces of the central portion by a silicone adhesive.

10. The valve of claim 1, wherein the first and second valve portions comprise perimeters at least partially surrounding an outer perimeter of the central valve portion.

11. The valve of claim 10, wherein the perimeters of the first and second valve portions contact one another adjacent the outer perimeter of the central valve portion.

12. The valve of claim 10, wherein the perimeters of the first and second valve portions are spaced apart from one another adjacent the outer perimeter of the central valve portion to provide a gap adjacent the outer perimeter.

13. The valve of claim 1, wherein the outer face of each of the first and second valve portions comprises a central region comprising the slit, an annular region surrounding the central region, and an outer perimeter, the annular region having a thickness thinner than the central region and the perimeter along the valve axis.

14. The valve of claim 1, wherein the passage comprises a circular opening extending between the central portion mating surfaces.

15. The valve of claim 14, further comprising an annular recess extending around the circular opening.

16. The valve of claim 1, wherein the central valve portion comprises a substantially circular outer perimeter smaller than outer perimeters of the outer valve portions.

17. The valve of claim 16, wherein one or both of the first and second outer valve portions comprise an outer lip extending axially from the outer valve portion mating surface that surrounds the outer perimeter of the central valve portion.

18. The valve of claim 1, wherein the central valve portion is formed from different material than one or both of the first and second outer valve portions.

19-24. (canceled)

25. The valve of claim 1, further comprising a lubricant applied to one or more of the first and second outer valve portions and the central valve portion.

26. The valve of claim 25, wherein the lubricant is applied to surfaces of the slits and the passage.

27. The valve of claim 25, wherein the lubricant comprises a swelling lubricant that is at least partially absorbed in material of the one or more of the first and second outer valve portions and the central valve portion.

28-29. (canceled)

30. A device for introducing one or more elongate devices into a patient's body, comprising:
a tubular member comprising a proximal end, a distal end sized for introduction into a patient's body, and a lumen extending between the proximal and distal ends;
a hub on the proximal end; and
a void valve received within the hub such that one or more medical devices may be introduced through the valve into the lumen, the valve comprising a central valve portion comprising central mating surfaces opposite one another and a passage extending between the central mating surfaces along a valve axis; and first and second outer valve portions, each outer valve portion comprising an outer valve portion mating surface, an outer face opposite the outer valve portion mating surface, and a slit extending substantially parallel to the valve axis between the outer valve portion mating surface and the outer face, the outer valve portion mating surface of the first and second outer valve portions attached to respective central mating surfaces of the central valve portion such that the slits are aligned with the passage on opposite sides of the central valve portion.

31. A method for making a valve for a hub of a sheath, catheter, or other tubular device, comprising:
forming a central valve portion comprising central portion mating surfaces opposite one another and a passage extending between the central portion mating surfaces along a valve axis;
forming first and second outer valve portions, each outer valve portion comprising an outer valve portion mating surface, an outer face opposite the outer valve portion mating surface, and a slit extending substantially parallel to the valve axis between the outer valve portion mating surface and the outer face; and
attaching the outer valve portion mating surface of the first and second outer valve portions to respective central portion mating surfaces of the central valve portion such that the slits are aligned with the passage on opposite sides of the central valve portion.

32-36. (canceled)
